(19) United States
(12) Patent Application Publication
(10) Pub. No.: US 2019/0016821 A1
(43) Pub. Date: Jan. 17, 2019

(54) STABILIZED PEPTIDE FRAGMENTS FROM PROTOCADHERIN FAT1 AS CANCER BIOMARKERS

(71) Applicant: TEMPLE UNIVERSITY OF THE COMMONWEALTH SYSTEM OF HIGHER EDUCATION, Philadelphia, PA (US)

(72) Inventor: Frank N. CHANG, Dresher, PA (US)

(73) Assignee: Temple University of the Commonwealth System of Higher Education, Philadelphia, PA (US)

(21) Appl. No.: 16/068,629

(22) PCT Filed: Jan. 5, 2017
(86) PCT No.: PCT/US2017/012242

§ 371 Date: Jul. 6, 2018

Related U.S. Application Data
(60) Provisional application No. 62/276,514, filed on Jan. 8, 2016.

(51) Int. Cl. C07K 16/32; G01N 33/574

(52) U.S. Cl. CPC C07K 16/32; G01N 2333/705; G01N 33/57434; G01N 33/5748

(57) ABSTRACT

An embodiment of the invention relates to the use of stabilized cancer peptide fragments derived from Protocadherin FAT1 for the diagnosis of cancers, particularly pancreatic cancer. A method for the detection of cancer, severity of cancer, and/or effectiveness of a therapeutic regimen includes detecting and/or measuring the amount of Protocadherin FAT1 peptide fragments present in the biological sample of a subject.

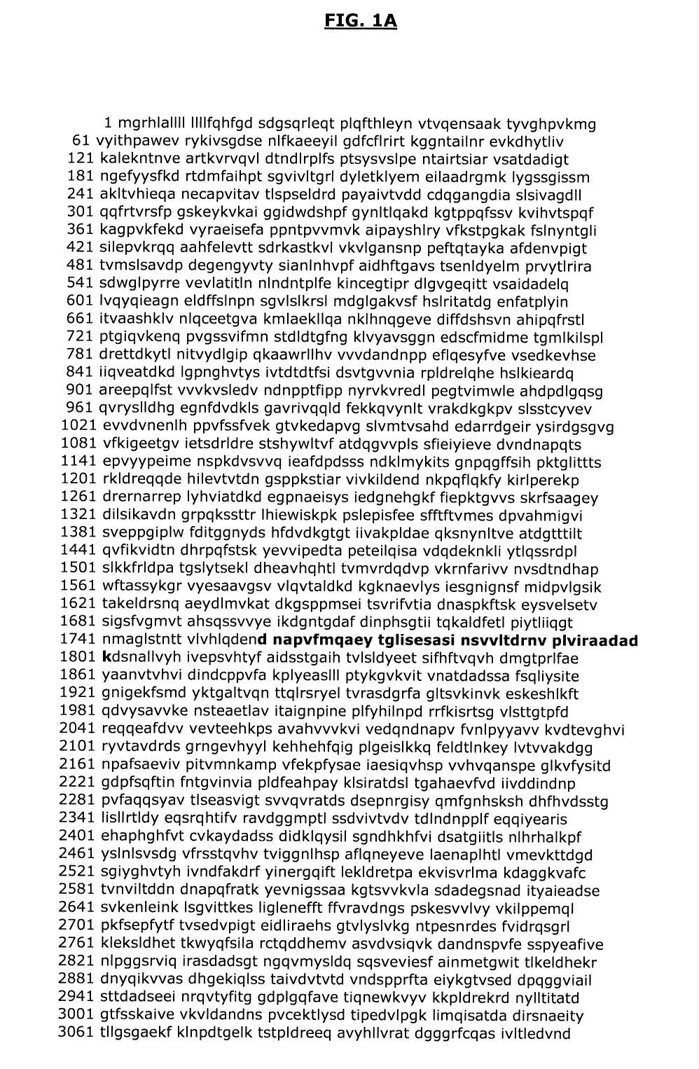

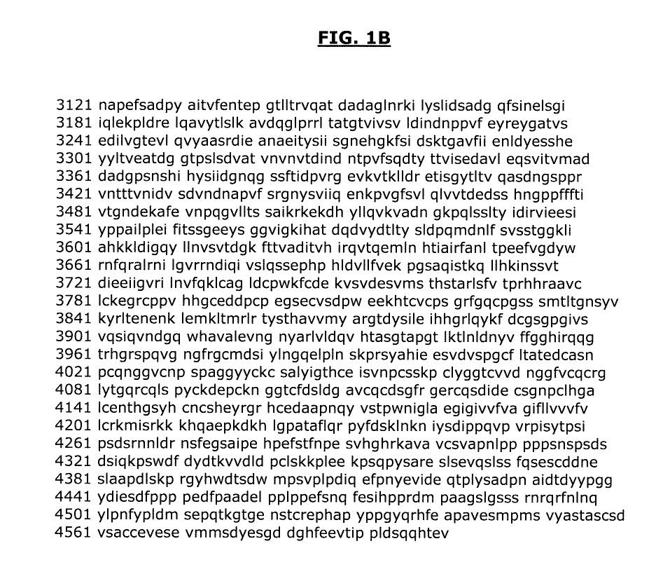

### CROSS REFERENCE TO RELATED APPLICATIONS

This is the national phase of International Application No. PCT/US2017/012242, filed 5 Jan. 2017, which claims priority to U.S. Provisional Application No. 62/276,514, filed 8 Jan. 2016. The disclosure of each of these applications is incorporated herein by reference in its entirety for all purposes.

### FIELD OF THE INVENTION

The present invention relates to the use of stabilized peptide fragments derived from Protocadherin FAT1 as early or late stage cancer biomarkers. Such biomarkers can be used to determine a diagnosis or prognosis, or to assess the effectiveness of a therapeutic intervention.

### BACKGROUND OF THE INVENTION

Biomarker research has exploded, primarily due to the use of proteomics approaches focusing on identifying differences in protein structure and abundance between diseased and normal states. Once identified, these biomarker proteins can be utilized for developing diagnostic tools, and because they are functional molecules, they are also more likely to be valid therapeutic targets.

The accessibility and presence of a large number of proteins in blood plasma make it an excellent matrix in which to search for new biomarkers. However, the estimated dynamic range of various protein concentrations in human serum is up to almost 10 orders of magnitude (Corthals et al., 2000), making the rapid identification of individual disease-associated proteins a tremendous analytical challenge. While total serum protein concentration is approximately 70-90 mg/ml, most useful biomarkers, such as cytokines and prostate specific antigen, are present in the nano to picogram range, and disease-specific changes can be expected to be incrementally small, especially in the early stages of disease (Merrell et al., 2004). Compounding these problems, many disease-specific proteins (e.g. cancer biomarker proteins) are degraded inside the cancer cell by proteolytic enzymes, generating peptide fragments that are subsequently released into the blood. Being low molecular weight in nature, these peptide fragments generally have a half-life of only about two to four hours and most of them are cleared from circulation by the kidney (Lowenthal et al., 2005).

In order to overcome challenges presented by low concentration and rapid turnover of potentially useful cancer peptide fragments, the albumin-associated fraction of proteins and peptides has been investigated as a source of useful new disease-specific biomarkers. Albumin, the most abundant plasma protein (40-50 mg/ml), functions as a scaffold for binding small molecules, lipids, peptides and proteins in the extracellular space. It has been found to form complexes with peptide hormones such as insulin and glucagon; bradykinin, serum amyloid A, interferons, the amino terminal peptide of HIV-1, gp41, and the 14-kDa fragment of streptococcal protein G, among others. Interestingly, it was found that a small percentage of the secreted peptide fragments from degraded cancer proteins have high affinity for serum albumin and form new serum albumin complexes which increase their half-life to about 19 days rather than 2 to 4 hours if they are freely circulating in the blood (Lowenthal et al., 2005). Thus, by their association with serum albumin to form complexes, the longevity of these cancer peptide fragments can be increased by more than 100-fold (Dennis et al., 2002). Due to its high affinity for such a diverse range of ligands, the serum albumin population is expected to be highly heterogeneous, most likely comprising hundreds of different albumin complexes.

Even the most widely used technology for protein separation, 2-dimensional polyacrylamide gel electrophoresis (2-D PAGE), introduced by O'Farrell (1975), cannot separate serum albumin complexes, as it is typically conducted under “denaturing” conditions. Additionally, 2-D PAGE has many other shortcomings including requiring large amounts of samples (about 50 to 100 μg of protein per experiment) and producing a rather streaky and mostly diffused profile when serum sample is analyzed. Furthermore, proteins separated by 2-D PAGE are required to be “blotted” or transferred onto blotting membranes such as polyvinylidene difluoride (PVDF) for Western blot analysis. The efficiency of protein blotting is also variable.

As described in WO 2011/008746, the present inventors developed a new electrophoresis procedure that separates serum protein complexes directly on the PVDF membrane, thus bypassing the cumbersome, time-consuming gel electrophoresis and its subsequent blotting steps (Chang and Yonan, 2008; Chang et al., 2009). The separation of albumin complexes in the present inventors' 2-D High Performance Liquid Electrophoresis (2-D HPLE) is based on their net charge or isoelectric points (pI). The association of a newly released cancer peptide fragment with a pre-existing albumin complex changes its pI and this new albumin complex migrates to a different location on the PVDF membrane, allowing its detection among hundreds of already present albumin complexes. Because it focuses on disease specific peptide fragments, the technique enables not only the identification of new cancer protein biomarkers, but also identifies the cancer peptide motifs within these proteins. When LC-MS/MS analysis is preceded by fraction separation using 2-D HPLE, its dynamic range is enhanced to the 10¹⁰ range required for detecting low copy number cancer biomarkers, a sensitivity that has not previously been achieved using other protein separation techniques.

In the United States, pancreatic cancer is the fourth leading cause of cancer-related death in both men and women. It is estimated by the National Cancer Institute that in 2014 more than 46,000 people in the United States will be diagnosed with pancreatic cancer and more than 39,000 will die of this disease. Pancreatic cancer incidence and mortality rates are higher in men than in women. African Americans also have higher rates of pancreatic cancer incidence and mortality than whites or other racial/ethnic groups.

Early stage pancreatic cancer is asymptomatic, and there is no routine screening test for pancreatic cancer. Because pancreatic cancer usually is diagnosed at an advanced stage, the survival rate is extremely low compared with those of many other cancer types. At this time, cancer of the pancreas can be cured only when it is found at an early stage (before it has spread) and only if surgery can completely remove the tumor. Standard treatments for pancreatic cancer include surgery, radiation therapy, chemotherapy, and targeted therapy. It is estimated that approximately $2.3 billion is spent in the United States each year on pancreatic cancer treatment. Serum biomarkers for early detection of pancreatic and other cancers are urgently needed and they will save lives.

Currently, only very limited reports on biomarkers for the detection of early stage cancers are available. People are regularly told to watch for early symptoms of cancer. However, by the time symptoms occur, many tumors have already grown quite large and may have metastasized. Moreover, many cancers such as pancreatic and prostate cancers have no symptoms. There remains a pressing need for biomarkers of early stage and late stage cancer to enable the detection, diagnosis, and treatment of cancer at its earliest stages of development, as well as its later stages of development.

### SUMMARY OF THE INVENTION

The present invention pertains to serum biomarkers for the diagnosis of cancer, such as pancreatic cancer, comprising stabilized cancer peptide fragments from the human Protocadherin FAT1 protein (also known as Protocadherin FAT1).

One embodiment of the invention provides a biomolecule (e.g., an antibody or antibody fragment) that is selective for a Protocadherin FAT1 peptide fragment, wherein the Protocadherin FAT1 peptide fragment comprises, consists essentially of, or consists of an amino acid sequence selected from the group consisting of SEQ ID NOS: 1-11. The biomolecule may be selective for a Protocadherin FAT1 peptide fragment having an amino acid sequence selected to from the group consisting of SEQ ID NOS: 1-11. The biomolecule may be used to determine, for example, whether a subject is predisposed to cancer, whether a subject has early stage cancer, or whether a subject has late state cancer.

Another embodiment of the present invention provides a method that comprises comparing the expression level of a Protocadherin FAT1 peptide fragment having an amino acid sequence selected from the group consisting of SEQ ID NOS: 1-11 in a first individual to the expression level in a healthy (cancer-free) individual. For example, the first individual may be suspected to suffer from cancer and/or may be susceptible to cancer. In another embodiment of the present invention a method comprises comparing the expression levels of a plurality of cancer peptide fragments having amino acid sequences selected from the group consisting of SEQ ID NOS: 1-11 in the first individual to the expression levels in a healthy (cancer-free) individual. In a more preferred embodiment of the method, an increase in the expression levels of said peptide fragment(s), compared to a healthy (cancer-free) individual, is indicative of cancer or the susceptibility to cancer.

According to particular embodiments, the accuracy of the diagnosis of cancer can be increased by analyzing combinations of multiple cancer peptides having amino acid sequences selected from the group consisting of SEQ ID NOS: 1-11. Thus, the method may comprises at least two, at least three, at least four, or at least five of the peptide fragments listed from the group consisting of SEQ ID NOS: 1-11.

In yet another embodiment, the present invention provides a method for assessing the severity or aggressiveness of cancer based on expression levels of a serum cancer peptide fragment, or a plurality of cancer peptide fragments, selected from the group consisting of SEQ ID NOS: 1-11. In another embodiment, the present invention provides a method for assessing the effectiveness of a therapeutic intervention for cancer based on expression levels of a serum cancer peptide fragment, or a plurality of cancer peptide fragments, selected from the group consisting of SEQ ID NOS: 1-11 before and during treatment.

Another embodiment of the invention provides a kit for diagnosing cancer in a subject, for determining whether a subject is predisposed to cancer, and/or for assessing the progression of cancer in a subject, the kit comprising one or more biomolecules (e.g., antibodies or antibody fragments), wherein each biomolecule is selective for a Protocadherin FAT1 peptide fragment that comprises, consists essentially of, or consists of an amino acid sequence selected from the group consisting of SEQ ID NOS: 1-11.

Another embodiment of the invention provides a method for determining whether cancer (early stage or late stage) is present in a subject and/or whether a subject is predisposed to cancer, the method comprising determining whether one or more Protocadherin FAT1 peptide fragments are present in a biological sample obtained from the subject, wherein each Protocadherin FAT1 peptide fragment comprises, consists essentially of, or consists of an amino acid sequence selected from the group consisting of SEQ ID NOS: 1-11; wherein said determining is performed by contacting the biological sample with one or more biomolecules selective for the one or more Protocadherin FAT1 peptide fragments and detecting whether binding occurs between the one or more Protocadherin FAT1 peptide fragments and the one or more biomolecules, wherein binding between the one or more Protocadherin FAT1 peptide fragments and the one or more biomolecules indicates the presence of one or more Protocadherin FAT1 peptide fragments in the biological sample; and wherein the presence of one or more Protocadherin FAT1 peptide fragments in the biological sample indicates that early or late stage cancer is present in the subject or that the subject is predisposed to cancer.

Another embodiment of the present invention provides a method for monitoring the progression of cancer in a subject comprising determining the amount of one or more Protocadherin FAT1 peptide fragments present in the biological sample at a first time point, determining the amount of one or more Protocadherin FAT1 peptide fragments present in the biological sample at one or more subsequent time points, and comparing the amount of the one or more Protocadherin FAT1 peptide fragments present in the biological sample at the one or more subsequent time points with the amount of the one or more Protocadherin FAT1 peptide fragments present in the biological sample at the first time point, wherein a higher amount of the one or more Protocadherin FAT1 peptide fragments at the one or more subsequent time points compared to the amount of the one or more Protocadherin FAT1 peptide fragments at the first time point indicates that the cancer has progressed since the first time point, and wherein a lower amount of the one or more Protocadherin FAT1 peptide fragments at the one or more subsequent time points compared to the amount of the one or more Protocadherin FAT1 peptide fragments at the first time point indicates that the cancer has regressed since the first time point, wherein each of the one or more Protocadherin FAT1 peptide fragments comprises, consists essentially of, or consists of an amino acid sequence selected from the group consisting of SEQ ID NOS: 1-11

Another embodiment of the present invention provides a method for treating cancer in a subject comprising administering to the subject an effective amount of a biomolecule that is selective for a Protocadherin FAT1 peptide fragment comprising, consisting essentially of, or consisting of an amino acid sequence selected from the group consisting of SEQ ID NOS: 1-11.

### DETAILED DESCRIPTION OF THE INVENTION

The applicants have discovered stabilized cancer peptide fragments, referred to herein as “Protocadherin FAT1 peptide fragments,” that are indicative of early stage cancer, late stage cancer, and/or a subject's predisposition for cancer. According to particular embodiments of the present invention, the stabilized cancer peptide fragments can be used as biomarkers for detecting early stage or late stage cancer in a subject and/or for detecting a subject's susceptibility to cancer. The cancer peptide fragments are derived from the Protocadherin FAT1 protein (i.e., they are fragments of the protein).

Even though many proteins are found to be over-produced in cancer cells (via either genomic or proteomic approaches) and constitute the so-called cancer biomarker protein repertoire, a vast majority of them have not been successfully used as serum biomarkers. One reason is because most of the reported cancer proteins are large molecules which are normally degraded to peptide fragments inside the cancer cell. As discussed herein, once released into the blood, these peptide fragments are subjected to further degradation with the resulting shorter peptide fragments later cleared from circulation by the kidneys (Lowenthal et al., 2005). Circulatory cancer peptide fragments have a short half-life of only about two to four hours which makes their detection highly variable and inconsistent when used as serum cancer biomarkers (Lowenthal et al., 2005). Compounding the problem further is the fact that “Sandwich ELISA” is normally used for their detection. In Sandwich ELISA, two antibodies directed against the same peptide (or protein) must be used. A capture antibody which recognizes a specific region of the peptide and a detection antibody recognizing a different epitope of the same peptide have to be generated. Due to the fact that it often requires a peptide sequence of 10 to 15 amino acids for producing a specific antibody, the circulating peptide will need to have at least 25 to 30 amino acids to be useful for generating both the capture and detection antibodies. Most circulatory cancer peptides are likely to be shorter than 25 amino acids and even if they are longer peptides, their short half-life (less than four hours) will not allow for their consistent detection either.

Contrary to the “transient” nature of many circulatory peptide fragments in the blood, stabilized peptide fragments from over-produced cancer proteins can be developed into new serum biomarkers. One way to find such stabilized peptide fragments is via their association with serum albumin. Only a very small fraction of peptide fragments originating from cancer proteins, if any, has high affinity for serum albumin and is sequestered by albumin. This association increases their blood half-life more than 100 fold over free circulating peptide fragments (Dennis et al., 2002). This sequestration by serum albumin makes cancer peptides stable and their detection very consistent rather than sporadic as in the case of free (unbound) circulatory peptides. Essentially, serum albumin acts as an affinity matrix for a limited number of cancer peptide fragments. A method for finding the stabilized (sequestered) cancer peptide fragments is to use a 2-D High Performance Liquid Electrophoresis (2-D HPLE) process which separates serum albumin complexes under non-denaturing conditions. 2-D HPLE technology described in WO 2011/008746 (which is incorporated herein by reference) can cleanly separate about 400 serum albumin complexes circulating in the blood; of these, about 250 albumin complexes are in the hydrophilic fraction and 150 in the hydrophobic fraction. The hydrophobic fraction of serum albumin complexes was obtained via partition using Triton-X114 (Bordier, 1981). The separation of serum albumin complexes on polyvinylidene fluoride (PVDF) membrane also prevents the separated albumin complexes from diffusion as would have occurred with proteins separated by a polyacrylamide gel. Mass spectrometric analysis (LC-MS/MS) of the newly generated albumin complex reveals both the amino acid composition of the sequestered peptide fragment and equally importantly, the cancer protein it derived from. Thus, unlike the commonly used genomic or proteomic approaches which can only identify protein cancer biomarkers, the 2-D HPLE can accomplish two purposes: 1) finding new cancer biomarker proteins and 2) discovering the stabilized peptide fragments from the cancer proteins that can be used as new serum cancer biomarkers. Using 2-D HPLE, the applicants discovered stabilized cancer peptide fragments from Protocadherin FAT1.

According to particular embodiments, the present invention provides a biomolecule that is selective for (i.e., that specifically binds to) a Protocadherin FAT1 peptide fragment. The Protocadherin FAT1 peptide fragment may comprise, consist essentially of, or consist of an amino acid sequence selected from the group consisting of SEQ ID NOS: 1-11. The biomolecule according to these embodiments may be, for example, an antibody (monoclonal or polyclonal) or an antibody fragment (e.g., a Fab fragment). For example, the biomolecule may be selected from the group consisting of a recombinant antibody, a recombinant monoclonal antibody, a polyclonal antibody, a humanized antibody and an antibody fragment.

According to particular embodiments, the biomolecule (e.g., antibody) is useful for diagnosing cancers that utilize Protocadherin FAT1 protein and/or for determining whether a subject is predisposed to cancers that utilize Protocadherin FAT1 protein.

According to particular embodiments, the biomolecule (e.g., antibody) is useful for diagnosing early stage or late stage cancer selected from the group consisting of pancreatic cancer, breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma. According to preferred embodiments, the biomolecule is useful for diagnosing early stage or late stage pancreatic cancer.

Another embodiment of the present invention provides an array comprising a plurality of the biomolecules, which can be used to diagnose early stage or late stage cancer and/or to determine whether a subject is predisposed to cancer.

Another embodiment of the present invention provides a composition comprising, consisting essentially of, or consisting of the biomolecule in a pharmaceutically acceptable carrier. Alternatively, a composition may include at least two biomolecules in a pharmaceutically acceptable carrier. As used herein, the term “pharmaceutically acceptable carrier” refers to a diluent, adjuvant, excipient, or vehicle with which a biomolecule (e.g., antibody) of the present invention is administered. Such carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when a compound is administered intravenously. Saline solutions and aqueous dextrose and glycerol solutions can also be employed as liquid carriers, particularly for injectable solutions. Suitable excipients include starch, glucose, lactose, sucrose, gelatin, silica gel, sodium stearate, glycerol monostearate, talc, sodium chloride, glycerol, propylene glycol, water, ethanol and the like. A pharmaceutically acceptable carrier can also include minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be included in a carrier. Methods for producing compounds in combination with carriers are known to those of skill in the art.

According to another embodiment, the present invention provides a kit for diagnosing early stage or late stage cancer in a subject and/or for determining whether a subject is predisposed to cancer, the kit comprising: one or more biomolecules (preferably an antibody or antibody fragment) wherein each biomolecule is selective for a Protocadherin FAT1 peptide fragment that comprises, consists essentially of, or consists of an amino acid sequence selected from the group consisting of SEQ ID NOS: 1-11. The kit is particularly useful for diagnosing early stage or late stage cancer and/or for determining whether a patient is predisposed to such cancers.

The kit may be used for diagnosing early stage or late stage cancer selected from the group consisting of pancreatic cancer, breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma. According to exemplary embodiments, the kit is useful for diagnosing early stage or late stage pancreatic cancer.

According to another embodiment of the present invention, a method for determining whether early stage or late stage cancer is present in a subject (or whether a subject is predisposed to cancer) comprises determining whether one or more Protocadherin FAT1 peptide fragments are present in a biological sample obtained from a subject, wherein each of the one or more Protocadherin FAT1 peptide fragments comprises, consists essentially of, or consists of any of SEQ ID NOS: 1-11.

The determining step is preferably performed by contacting the biological sample with one or more biomolecules (preferably an antibody or antibody fragment) selective for the one or more BRCA1 and/or BRCA2 peptide fragments and detecting whether binding occurs between the one or more Protocadherin FAT1 peptide fragments and the one or more biomolecules. The one or more biomolecules bind to an epitope that is present on the one or more Protocadherin FAT1 peptide fragments that each biomolecule is specific for (e.g., an antibody specific for a Protocadherin FAT1 peptide fragment having SEQ ID NO: 1 binds to an epitope on said Protocadherin FAT1 peptide fragment and/or an antibody specific for a Protocadherin FAT1 peptide having SEQ ID NO: 12 binds to an epitope on said Protocadherin FAT1 peptide fragment, etc.). Binding between the one or more Protocadherin FAT1 peptide fragments and the one or more biomolecules indicates the presence of one or more Protocadherin FAT1 peptide fragments in the biological sample. The presence of one or more Protocadherin FAT1 peptide fragments in the biological sample indicates that cancer is present in the subject or that the subject is predisposed to cancer.

According to an alternative embodiment, the method comprises comparing the amount of the one or more Protocadherin FAT1 peptide fragments in the biological sample to the amount of one or more Protocadherin FAT1 peptide fragments in a biological sample from a subject that is known to be cancer-free, wherein a higher amount of one or more Protocadherin FAT1 peptide fragments in the biological sample compared to the amount of one or more Protocadherin FAT1 peptide fragments in the biological sample from the cancer-free subject indicates that early stage or late stage cancer is present in the subject or that the subject is predisposed to cancer.

The biological sample may comprise, for example, serum, plasma, cells of cancer tissues, fluids and the like. Non-limiting examples of fluids include serum, plasma, cells of cancer tissues, fluids such as blood, cerebro-spinal fluid, feces, gingival crevicular fluid, lachrymal fluid, lymph, perspiration, mammary gland secretions, mucus, saliva, semen, sputum, synovial fluid, tears, urine, vaginal secretions, and vitreous humor, preferably blood and serum. According to particular embodiments, the biological sample is from cells of pancreatic tissue or cells of an pancreatic tumor. According to additional embodiments, suitable biological samples to be analyzed for the presence or absence of a biomarker can be serum, plasma, pancreatic juice, cells of a pancreatic tumor, or cells of pancreatic tissue. Cells from pancreatic tissue can be obtained, for example, by ERCP, secretin stimulation, fine-needle aspiration, cytologic brushings and large-bore needle biopsy.

The step of detecting whether binding occurs between the one or more Protocadherin FAT1 peptide fragments and the one or more biomolecules may be performed by using an ELISA, preferably an ELISA that utilizes only one antibody instead of two or more antibodies, such as a peptide ELISA or a competitive ELISA.

According to particular embodiments, the subject has not been diagnosed with cancer prior to performing said method. Alternatively, the subject has not been diagnosed with late stage cancer and the presence of one or more Protocadherin FAT1 peptide fragments in the biological sample indicates the presence of early stage cancer in the subject. As used herein, “early stage cancer” includes any pre-cancerous state prior to late stage cancer, including but not limited to benign conditions (any non-cancerous abnormality that has the potential to develop into late stage cancer), conditions prior to invasive carcinoma, and/or conditions prior to the development of a cancerous tumor. With regard to breast cancer, for example, “early stage cancer” includes any pre-cancerous state prior to stage I, stage II, stage III, or stage IV cancer. Examples of early stage breast cancer include benign conditions (e.g., non-proliferative lesions, proliferative lesions without atypia, and proliferative lesions with atypia), dysplasia, and/or carcinoma in situ. With regard to cancers other than breast cancer (e.g., colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, or stomach adenocarcinoma), “early stage cancer” includes any pre-cancerous state prior to late stage cancer, such as those that correspond to stage I, stage II, stage III, or stage IV in breast cancer.

As used herein, “cancer-free” refers to a subject who is free of early stage and late stage cancer, or to tissue of a subject that is free of early stage and late stage cancer. A subject as used herein is preferably an animal, including but not limited to mammals, and most preferably human.

According to another embodiment of the present invention, a method for treating early stage or late stage cancer in a subject comprises administering to the subject an effective amount of a biomolecule (preferably an antibody or antibody fragment) that is selective for a peptide fragment comprising, consisting essentially of, or consisting of an amino acid sequence selected from SEQ ID NOS: 1-11. The method may inhibit or arrest the progression of cancer in the subject and/or inhibit or arrest the progression of early stage cancer to late stage cancer.

As used herein, the term “effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent the progression of cancer.

Methods for administering an antibody specific for a peptide are known in the art. Antibodies are commonly used as therapeutic compounds, and those of ordinary skill in the art would be able to discern appropriate dosages and methods of use for the antibodies of the present invention. For example, the administering physician can easily determine optimum dosages, dosing methodologies and repetition rates. According to particular embodiments, dosage is from about 0.001 μg to about 100 g per kg of a subject's body weight. The treating physician can estimate repetition rates for dosing based on measured residence times and concentrations of the antibody in bodily fluids or tissues.

According to alternative embodiments, the method may comprise administering to the subject an effective amount of two or more biomolecules selective for a peptide fragment comprising, consisting essentially of, or consisting of an amino acid sequence selected from SEQ ID NOS: 1-11. The biomolecule(s) may be provided in a pharmaceutical carrier.

Another embodiment of the present invention provides a method for monitoring the progression of early stage or late stage cancer in a subject (e.g., for assessing the effectiveness of a treatment regime or therapeutic agent) comprising determining the amount of one or more Protocadherin FAT1 peptide fragments present in the biological sample at a first time point, determining the amount of one or more Protocadherin FAT1 peptide fragments present in the biological sample at one or more subsequent time points, and comparing the amount of the one or more Protocadherin FAT1 peptide fragments present in the biological sample at the one or more subsequent time points with the amount of the one or more Protocadherin FAT1 peptide fragments present in the biological sample at the first time point. A higher amount of the one or more Protocadherin FAT1 peptide fragments at the one or more subsequent time points compared to the amount of the one or more Protocadherin FAT1 peptide fragments at the first time point indicates that the cancer has progressed (the amount or severity of cancer has increased) since the first time point. A lower amount of the one or more Protocadherin FAT1 peptide fragments at the one or more subsequent time points compared to the amount of the one or more Protocadherin FAT1 peptide fragments at the first time point indicates that the cancer has regressed since the first time point (the amount or severity of cancer has decreased). Each of the one or more Protocadherin FAT1 peptide fragments comprises, consists essentially of, or consists of an amino acid sequence selected from SEQ ID NOS: 1-11.

The step(s) of determining the amount of one or more Protocadherin FAT1 peptide fragments at the first time point and the one or more subsequent time points is preferably performed by contacting the biological sample with one or more biomolecules (preferably one or more antibodies or antibody fragments) selective for the one or more Protocadherin FAT1 peptide fragments and detecting whether binding occurs between the one or more Protocadherin FAT1 peptide fragments and the one or more biomolecules, wherein the one or more biomolecules bind to an epitope that is present on the one or more Protocadherin FAT1 peptide fragments. The step(s) of determining may be performed by using an ELISA, preferably an ELISA that utilizes only one antibody instead of two or more antibodies, such as a peptide ELISA or a competitive ELISA.

According to particular embodiments, the first time point is prior to a treatment regimen and the one or more subsequent time points are during or after the treatment regimen, wherein the method monitors the effectiveness of the treatment regimen over time.

According to another embodiment of the present invention, a method of producing antibodies comprises administering a Protocadherin FAT1 peptide fragment to an immunologically competent host in an amount effective to cause the host to generate antibodies specific for the Protocadherin FAT1 peptide fragment, respectively, wherein the peptide fragment has an amino acid sequence that comprises, consists essentially of, or consists of a sequence selected from SEQ ID NOS: 1-11, and recovering antibodies from the host.

According to an embodiment, an antibody that binds specifically to a cancer polypeptide comprising, consisting essentially of, or consisting of a cancer peptide fragment having an amino acid sequence selected from SEQ ID NOS: 1-11 can be used to inhibit the growth of the cancer cell. A cancer biomarker array, comprising a plurality of antibodies, wherein each antibody binds specifically to a cancer polypeptide fragment comprising, consisting essentially of, or consisting of an amino acid sequence selected from SEQ ID NOS: 1-11 can be used to assess the presence, cancer severity and metastatic potential of different cancers.

As discussed herein, antibodies raised against Protocadherin FAT1 peptide fragments that comprise, consist essentially of, or consist of amino acid sequences selected from SEQ ID NOS: 1-11 are highly specific “peptide antibodies” recognizing only a small region of the Protocadherin FAT1 cancer protein (rather than the entire protein). Because the antibodies are raised against a very small region (or unique region) and not to the whole protein, they will be highly specific and will generally not be able to cross-react with other protein in the body. Antibodies directed against an entire cancer protein will likely miss a specific cancer peptide motif and therefore render them ineffective (or much less effective) in detecting a particular cancer peptide fragment. Furthermore, therapeutic drugs targeting a short cancer peptide (or cancer peptide motif) sequence will be highly specific and will be expected to have much fewer side effects, if any. According to particular embodiments, the biomolecules of the present invention are specific for (i.e., are capable of detecting in a biological sample) peptides consisting essentially of, or consisting of, an amino acid sequence selected from SEQ ID NOS: 1-11, but they are not specific for (i.e., are not capable of detecting in a biological sample) the entire protein from which the fragment originates.

### EXAMPLES

### Example 1. Stabilized Cancer Peptide Biomarkers from Protocadherin FAT1

The applicants have discovered a 42-amino acid fragment DNAPVFMQAEYTGLISESASINSWLTDRNVPLVIRAADADK (SEQ ID NO: 1) that was found to be sequestered in a serum albumin complex from pancreatic cancer patients' serum. It is derived from amino acids 1,760 to 1,801 of the 4,600 amino acid Protocadherin FAT1 (SEQ ID NO: 12).

It should be pointed out that even though Protocadherin FAT1 has been reported to be over-produced in many cancers, and its secreted fragments have been suggested as cancer biomarkers, this particular 42-amino acid peptide fragment has not previously been reported as a cancer biomarker. Without the knowledge of a stabilized peptide sequence, the probability of finding this specific fragment from Protocadherin FAT 1 is extremely low. Even though antibodies against Protocadherin FAT 1 are commercially available, they are directed against much longer peptides that are most likely not sequestered. These commercially available antibodies cannot detect specifically the stable circulatory peptide fragment and will be of little value in detecting the cancer biomarker described herein.

This peptide sequence, DNAPVFMQAEYTGLISESASINSWLTDRNVPLVIRAADADK (SEQ ID NO: 1), may be used to generate cancer peptide-specific biomolecules (e.g., antibodies or antibody fragments). The antibodies can be either polyclonal or monoclonal. Besides being used to detect the stabilized peptide fragment, the antibodies can also be used to detect the over-production of Protocadherin FAT1 in cancer cells via immunohistochemical (IHC) staining. For example, the sequence used to produce a biomolecule (e.g., an antibody) may contain between about 6 to about 20 amino acids, although other lengths may also be used.

Several alternative strategies may be used to produce a biomolecule (e.g., an antibody) against SEQ ID NO: 1. As used herein, a biomolecule that is selective for SEQ ID NO: 1 is a biomolecule that is selective for a portion or fragment of SEQ ID NO: 1, or that is selective for the entire SEQ ID NO: 1. For example, a biomolecule that is selective for a portion or fragment of SEQ ID NO: 1 may be a biomolecule that is selective for a peptide fragment that comprises, consists essentially of, or consists of a peptide sequence within SEQ ID NO: 1 (i.e., a portion or fragment of SEQ ID NO: 1), wherein the peptide fragment is 10-15 amino acids or 6-20 amino acids in length (e.g., SEQ ID NO: 3 or SEQ ID NO: 4).

The long sequence of SEQ ID NO: 1 makes it suitable for developing Sandwich ELISA, which generally utilizes two antibodies. According to particular embodiments, two biomolecules (e.g., antibodies) are raised against two different peptide sequences within SEQ ID NO: 1, respectively. Stated another way, a first biomolecule (e.g. antibody) is specific for a first peptide sequence within SEQ ID NO: 1, and a second biomolecule (e.g. antibody) is specific for a second peptide sequence within SEQ ID NO: 1, wherein the first peptide sequence and the second peptide sequence are different from each other, but each peptide sequence is found within SEQ ID NO: 1. For example, the two peptide sequences may be adjacent to each other within SEQ ID NO: 1. Preferably, each of the two biomolecules is specific for a peptide sequence within SEQ ID NO: 1 that is 10 to 15 amino acids in length, or 6 to 20 amino acids in length.

According to additional embodiments, only one biomolecule (e.g., antibody) is raised against a peptide fragment that comprises, consists essentially of, or consists of a peptide sequence within SEQ ID NO: 1, wherein the peptide sequence is preferably 10 to 15 amino acids in length, or 6 to 20 amino acids in length.

One example for a first antibody to be used for a Sandwich ELISA is XXX DNAPVFMQAEYTGLXXX (SEQ ID NO: 2), where X at positions 1-3 and 18-20 may be any naturally-occurring or artificial amino acid and up to six of the amino acids may be absent (i.e., any one or all of the X amino acids may be present or absent). A cysteine residue is preferably added to these peptide sequences, for example DNAPVFMQAEYTGLC (SEQ ID NO: 3) for conjugational purposes to produce the antibody. The cysteine residue can also be added to the N-terminal end, which becomes CDNAPVFMQAEYTGL (SEQ ID NO: 4). Another example of the peptide sequence that may be used for the production of a biomolecule (e.g., antibody) for SEQ ID NO: 1 is CXXDNAPVFMQAEYTGLXXX (SEQ ID NO: 5) where X at positions 2-3 and 18-20 may be any naturally-occurring or artificial amino acid and up to five of the amino acids may be absent (i.e., any one or all of the X amino acids may be present or absent). Another example of the peptide sequence that may be used for the production of a biomolecule (e.g., antibody) for SEQ ID NO: 1 is XXXDNAPVFMQAEYTGLXXC (SEQ ID NO: 6) where X at positions 1-3 and 18-19 may be any naturally-occurring or artificial amino acid and up to five of the amino acids may be absent (i.e., any one or all of the X amino acids may be present or absent).

The Sandwich ELISA generally utilizes two antibodies. The amino acid sequence for producing the second antibody can be selected from the 42 amino acids provided they are preferably separated from those selected for the production of the first antibody to minimize the interference between the two antibodies. One example of the peptide selected for antibody production is XXXDRNVPLVIRAADADKXXX (SEQ ID NO: 7), where X at positions 1-3 and 19-21 may be any naturally-occurring or artificial amino acid and up to six of the amino acids may be absent (i.e., any one or all of the X amino acids may be present or absent). A cysteine residue is preferably added to these peptide sequences, for example, CDRNVPLVIRAADADK (SEQ ID NO: 8) or DRNVPLVIRAADADKC (SEQ ID NO: 9) for conjugational purposes to produce the antibody. Another example of the peptide sequence that may be used for the production of a biomolecule (e.g., antibody) for SEQ ID NO: 1 is CXXDRNVPLVIRAADADKXXX (SEQ ID NO: 10) where X at positions 2-3 and 19-21 may be any naturally-occurring or artificial amino acid and up to five of the amino acids may be absent (i.e., any one or all of the X amino acids may be present or absent). Another example of the peptide sequence that may be used for the production of a biomolecule (e.g., antibody) for SEQ ID NO: 1 is XXXDRNVPLVIRAADADKXXC (SEQ ID NO: 11) where X at positions 1-3 and 19-20 may be any naturally-occurring or artificial amino acid and up to five of the amino acids may be absent (i.e., any one or all of the X amino acids may be present or absent). Preferably, a peptide sequence of SEQ ID NOS. 2, 5, 6, 7, 10 or 11 has 10 to 15 amino acids, or 6 to 20 amino acids.

The discovery of a stabilized Protocadherin FAT1 peptide fragment circulating in the blood is significant. Besides pancreatic cancer, mutations in Protocadherin FAT1 gene have been found in other cancers including lung, breast, head and neck cancers, and glioma. The serum level of this Protocadherin FAT1 peptide fragment may be used as a biomarker for the presence of these cancers. Additionally, measurement of the levels of the stabilized Protocadherin FAT1 peptide fragment before and during treatment would indicate whether or not a therapeutic intervention is working.

Biomolecules (e.g., antibodies) specific for a peptide fragment having an amino acid sequence comprising, consisting essentially of, or consisting of a sequence selected from the group consisting of SEQ ID NOS. 1-11 may be useful for the detection of cancers and/or for assessing the progression of cancer and/or for assessing a treatment regimen, wherein the cancer may be selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma. According to particular embodiments, biomolecules specific for a peptide fragment having an amino acid sequence comprising, consisting essentially of, or consisting of a sequence selected from the group consisting of SEQ ID NOS. 1-11 may be useful for the detection and/or assessment of cancers other than breast cancer, such as colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, or stomach adenocarcinoma. According to particular embodiments, biomolecules specific for a peptide fragment having an amino acid sequence comprising, consisting essentially of, or consisting of a sequence selected from the group consisting of SEQ ID NOS. 1-11 may be useful for the detection and/or assessment of pancreatic cancer.

For any of the peptide sequences described herein (including all those sequences described throughout the Examples), it may be preferable to add an acetyl group to the N-terminal amino acid and/or to convert the C-terminal amino acid into an amide.

### Example 2

To quantify a serum cancer peptide fragment level, a “Peptide ELISA” or Competitive ELISA, rather than Sandwich ELISA, may be used. An embodiment of a protocol for a “Competitive ELISA” according to the present invention, using a single antibody against the peptide fragment, is provided below.

Experiments were carried out in triplicates. Two strip frames for inserting appropriate number of wells were prepared and name “plate A” and “plate B”. Wells for both standards and samples on plate A were coated overnight with 300 μl of 1% bovine serum albumin (BSA) in PBS (pH7.4), at 4° C. Wells for both standards and samples on plate B were coated with 200 μl of peptide solution containing 100 ng of peptide in sodium carbonate buffer (pH 9.6), overnight at 4° C. 110 μl of standard solution ranging from 1, 2, 4, 6, 8, 16, 20, and 40 ng/ml and sample solutions to be analyzed were added to the BSA-coated plates. Both the samples and standards were in TSBT high salt (Tris-buffered saline with 0.05% Tween-20 and 0.5 M NaCl). After incubating for 30 min at room temperature, 110 μl of biotinylated anti-peptide antibody was added. The antibody was diluted 1:100,000 (1 μl antibody+99 μl TBST-High salt, take 20 μl and dilute into 20 ml TBST high salt.). After addition of the antibody solution, the final concentrations of standard solution become 0.5, 1, 2, 3, 4, 8, 10 and 20 ng/ml. The wells were shaken overnight at 4° C.

The next day, the unoccupied space from the peptide coated wells was blocked by coating them with 1% BSA by shaking at room temperature for 30 min. The wells were washed 5 times in 300 μl TBST high salt. 200 μl of antigen-antibody mixture from the BSA coated plate was transferred to peptide coated plate and shaking at room temperature for 1.5 h. This is followed by washing with 300 μl TBST high salt 5 times. Streptavidin-HRP (1:54000 dilution in TBST high salt) was then added to the well (200 μl/well). After shaking at room temperature for 2 h, the wells were washed with 300 μl TBST high salt 5 times. This was followed by adding 100 μl TMB (tetramethylbenzidine, Pierce Chemical, Rockford, Ill., USA). The wells were shaken at room temperature until O.D. 450 of TBST control (0 ng/ml in the standard curve) reaches 1.0 M HCl (50 μl/well) was then added to the wells to stop the reaction. After reading at O.D.450, Prism 5.0 was used to generate the standard curve (one site-total function) and to calculate peptide concentrations of unknown samples.

The above Competitive ELISA can be used to quantify the serum level of stabilized peptide fragments of the present invention before treatment and during the treatment to assess the effectiveness of a therapeutic agent.

Although the present invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the appended claims.

1. A biomolecule that is selective for a Protocadherin FAT1 peptide fragment having an amino acid sequence selected from the group consisting of SEQ ID NOS: 1-11.

2. The biomolecule of claim 1, wherein the biomolecule is an antibody or an antibody fragment.

3. The biomolecule of claim 1, wherein the biomolecule is a monoclonal antibody or a polyclonal antibody.

4. The biomolecule of claim 1, wherein the biomolecule is selected from the group consisting of a recombinant antibody, a recombinant monoclonal antibody, a polyclonal antibody, a humanized antibody and an antibody fragment.

5. The biomolecule of claim 1, wherein the biomolecule is useful for diagnosing cancers that utilize Protocadherin FAT1 protein.

6. The biomolecule of claim 1, wherein the biomolecule is useful for determining whether a subject is predisposed to cancers that utilize Protocadherin FAT1 protein.

7. The biomolecule of claim 1, wherein the biomolecule is useful for diagnosing cancer selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma.

8. The biomolecule of claim 1, wherein the biomolecule is useful for diagnosing pancreatic cancer.

9. The biomolecule of claim 1, wherein the biomolecule is useful for determining whether a subject is predisposed to pancreatic cancer.

10. A composition comprising the biomolecule of claim 1 in a pharmaceutically acceptable carrier.

11. A composition comprising at least two biomolecules of claim 1 in a pharmaceutically acceptable carrier.

12. An array comprising a plurality of biomolecules of claim 1.

13. A kit for diagnosing cancer in a subject, for determining whether a subject is predisposed to cancer, and/or for assessing the progression of cancer in a subject, the kit comprising:
one or more biomolecules,
wherein each biomolecule is selective for a Protocadherin FAT1 peptide fragment having an amino acid sequence selected from the group consisting of SEQ ID NOS: 1-11.

14. The kit of claim 13, wherein the kit is useful for diagnosing cancers that utilize Protocadherin FAT1 protein.

15. The kit of claim 13, wherein the kit is useful for diagnosing cancer selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma.

16. The kit of claim 13, wherein the kit is useful for diagnosing pancreatic cancer.

17. The kit of any of claim 13, wherein each biomolecule is an antibody or an antibody fragment.

18. The kit of claim 13, wherein each biomolecule is a monoclonal antibody or a polyclonal antibody.

19. The kit of claim 13, wherein each biomolecule is selected from the group consisting of a recombinant antibody, a recombinant monoclonal antibody, a polyclonal antibody, a humanized antibody and an antibody fragment.

20. A method for determining whether cancer is present in a subject and/or whether a subject is predisposed to cancer, the method comprising:
determining whether one or more Protocadherin FAT1 peptide fragments are present in a biological sample obtained from the subject, wherein each Protocadherin FAT1 peptide fragment has an amino acid sequence selected from the group consisting of SEQ ID NOS: 1-11;
wherein said determining is performed by contacting the biological sample with one or more biomolecules selective for the one or more Protocadherin FAT1 peptide fragments and detecting whether binding occurs between the one or more Protocadherin FAT1 peptide fragments and the one or more biomolecules, wherein binding between the one or more Protocadherin FAT1 peptide fragments and the one or more biomolecules indicates the presence of one or more Protocadherin FAT1 peptide fragments in the biological sample; and
wherein the presence of one or more Protocadherin FAT1 peptide fragments in the biological sample indicates that cancer is present in the subject or that the subject is predisposed to cancer.

21. The method of claim 20 further comprising obtaining the biological sample from the subject.

22. The method of claim 20 further comprising comparing the amount of the one or more Protocadherin FAT1 peptide fragments in the biological sample to the amount of one or more Protocadherin FAT1 peptide fragments in a biological sample from a cancer-free subject, wherein a higher amount of one or more Protocadherin FAT1 peptide fragments in the biological sample compared to the amount of one or more Protocadherin FAT1 peptide fragments in the biological sample from the cancer-free subject indicates that cancer is present in the subject or that the subject is predisposed to cancer.

23. The method of claim 20, wherein the biological sample is plasma or serum.

24. The method of claim 20, wherein the cancer is selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma.

25. The method of claim 20, wherein the cancer is pancreatic cancer.

26. The method of claim 25, wherein the biological sample is from cells of pancreatic tissue or cells of a pancreatic tumor.

27. The method of claim 20, wherein each biomolecule is an antibody or an antibody fragment.

28. The method of claim 20, wherein each biomolecule is a monoclonal antibody or a polyclonal antibody.

29. The method of claim 20, wherein each biomolecule is selected from the group consisting of a recombinant antibody, a recombinant monoclonal antibody, a polyclonal antibody, a humanized antibody and an antibody fragment.

30. The method of claim 20, wherein detecting whether binding occurs between the one or more Protocadherin FAT1 peptide fragments and the one or more biomolecules is performed by using an ELISA.

31. The method of claim 20, wherein detecting whether binding occurs between the one or more Protocadherin FAT1 peptide fragments and the one or more biomolecules is performed by using a peptide ELISA or a competitive ELISA.

32. The method of claim 20, wherein the subject has not been diagnosed with cancer prior to performing said method.

33. The method of claim 20, wherein the subject has not been diagnosed with late stage cancer and the presence of one or more Protocadherin FAT1 peptide fragments in the biological sample indicates the presence of early stage cancer in the subject.

34. A method for treating cancer in a subject comprising administering to the subject an effective amount of a biomolecule that is selective for a peptide fragment having an amino acid sequence selected from the group consisting of SEQ ID NOS: 1-11.

35. The method of claim 34, wherein the method inhibits or arrests the progression of cancer in the subject.

36. The method of claim 34, wherein the method inhibits or arrests the progression of early stage cancer to late stage cancer.

37. The method of claim 34, comprising administering to the subject an effective amount of two or more biomolecules selective for a peptide fragment having an amino acid sequence selected from the group consisting of SEQ ID NOS: 1-11.

38. The method of claim 34, comprising administering a pharmaceutical composition to the subject, wherein the pharmaceutical composition comprises the biomolecule in a pharmaceutical carrier.

39. The method of claim 34, wherein the biomolecule is an antibody or an antibody fragment.

40. The method of claim 34, wherein the biomolecule is a monoclonal antibody or a polyclonal antibody.

41. The method of claim 34, wherein the biomolecule is selected from the group consisting of a recombinant antibody, a recombinant monoclonal antibody, a polyclonal antibody, a humanized antibody and an antibody fragment.

42. The method of claim 34, wherein the cancer is selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma.

43. The method of claim 34, wherein the cancer is pancreatic cancer.

44. A method for monitoring the progression of cancer in a subject comprising:
determining the amount of one or more Protocadherin FAT1 peptide fragments present in the biological sample at a first time point,
determining the amount of one or more Protocadherin FAT1 peptide fragments present in the biological sample at one or more subsequent time points, and
comparing the amount of the one or more Protocadherin FAT1 peptide fragments present in the biological sample at the one or more subsequent time points with the amount of the one or more Protocadherin FAT1 peptide fragments present in the biological sample at the first time point,
wherein a higher amount of the one or more Protocadherin FAT1 peptide fragments at the one or more subsequent time points compared to the amount of the one or more Protocadherin FAT1 peptide fragments at the first time point indicates that the cancer has progressed since the first time point, and
wherein a lower amount of the one or more Protocadherin FAT1 peptide fragments at the one or more subsequent time points compared to the amount of the one or more Protocadherin FAT1 peptide fragments at the first time point indicates that the cancer has regressed since the first time point,
wherein each of the one or more Protocadherin FAT1 peptide fragments has an amino acid sequence selected from the group consisting of SEQ ID NOS: 1-11.

45. The method of claim 44, wherein determining the amount of one or more Protocadherin FAT1 peptide fragments at the first time point and the one or more subsequent time points is performed by contacting the biological sample with one or more biomolecules selective for the one or more Protocadherin FAT1 peptide fragments and detecting whether binding occurs between the one or more Protocadherin FAT1 peptide fragments and the one or more biomolecules.

46. The method of claim 44 further comprising obtaining a biological sample from the subject.

47. The method of claim 44, wherein the first time point is prior to a treatment regimen and the one or more subsequent time points are during or after the treatment regimen, wherein the method monitors the effectiveness of the treatment regimen over time.

48. The method of claim 44, wherein the biomolecule is an antibody or an antibody fragment.

49. The method of claim 44, wherein the biomolecule is a monoclonal antibody or a polyclonal antibody

50. The method of claim 44, wherein the biomolecule is selected from the group consisting of a recombinant antibody, a recombinant monoclonal antibody, a polyclonal antibody, a humanized antibody and an antibody fragment.

51. The method of claim 44, wherein the cancer is selected from the group consisting of breast cancer, ovarian cancer, colon cancer, endometrial carcinoma, esophagus squamous cell carcinoma, glioma, hepatocellular carcinoma, infiltrating ductal breast carcinoma, larynx cancer, lung squamous cell carcinoma, melanoma, mucinous cystadenocarcinoma of ovary, pancreatic cancer, prostate cancer, renal cell carcinoma, small bowel malignant stromal tumor, and stomach adenocarcinoma.

52. The method of claim 44, wherein the cancer is pancreatic cancer.

53. A method of producing antibodies comprising:
administering a Protocadherin FAT1 peptide fragment to an immunologically competent host in an amount effective to cause the host to generate antibodies specific for the Protocadherin FAT1 peptide fragment, wherein the peptide fragment has an amino acid sequence selected from SEQ ID NOs: 1-11, and recovering antibodies from the host.


SEQUENCE LISTING (12 sequences)

SEQ ID NO: 1 - protein, 42 aa, Homo sapiens
DNAPVFMQAEYTGLISESASINSVVLTDRNVPLVIRAADADK

SEQ ID NO: 2 - protein, 20 aa, Homo sapiens
  misc_feature (1)..(3): Xaa can be any naturally occurring amino acid
  misc_feature (18)..(20): Xaa can be any naturally occurring amino acid
XXXDNAPVFMQAEYTGLXXX

SEQ ID NO: 3 - protein, 15 aa, Homo sapiens
DNAPVFMQAEYTGLC

SEQ ID NO: 4 - protein, 15 aa, Homo sapiens
CDNAPVFMQAEYTGL

SEQ ID NO: 5 - protein, 20 aa, Homo sapiens
  misc_feature (2)..(3): Xaa can be any naturally occurring amino acid
  misc_feature (18)..(20): Xaa can be any naturally occurring amino acid
CXXDNAPVFMQAEYTGLXXX

SEQ ID NO: 6 - protein, 20 aa, Homo sapiens
  misc_feature (1)..(3): Xaa can be any naturally occurring amino acid
  misc_feature (18)..(19): Xaa can be any naturally occurring amino acid
XXXDNAPVFMQAEYTGLXXC

SEQ ID NO: 7 - protein, 21 aa, Homo sapiens
  misc_feature (1)..(3): Xaa can be any naturally occurring amino acid
  misc_feature (19)..(21): Xaa can be any naturally occurring amino acid
XXXDRNVPLVIRAADADKXXX

SEQ ID NO: 8 - protein, 16 aa, Homo sapiens
CDRNVPLVIRAADADK

SEQ ID NO: 9 - protein, 16 aa, Homo sapiens
DRNVPLVIRAADADKC

SEQ ID NO: 10 - protein, 21 aa, Homo sapiens
  misc_feature (2)..(3): Xaa can be any naturally occurring amino acid
  misc_feature (19)..(21): Xaa can be any naturally occurring amino acid
CXXDRNVPLVIRAADADKXXX

SEQ ID NO: 11 - protein, 21 aa, Homo sapiens
  misc_feature (1)..(3): Xaa can be any naturally occurring amino acid
  misc_feature (19)..(20): Xaa can be any naturally occurring amino acid
XXXDRNVPLVIRAADADKXXC

SEQ ID NO: 12 - protein, 4600 aa, Homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGGIDWDSHPFGYNLTLQAKDKGTPPQFSSVKVIHVTSPQFKAGPVKFEKDVYRAEISEFAPPNTPVVMVKAIPAYSHLRYVFKSTPGKAKFSLNYNTGLISILEPVKRQQAAHFELEVTTSDRKASTKVLVKVLGANSNPPEFTQTAYKAAFDENVPIGTTVMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDFFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSIVTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDARRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYHVIATDKDEGPNAEISYSIEDGNEHGKFFIEPKTGVVSSKRFSAAGEYDILSIKAVDNGRPQKSSTTRLHIEWISKPKPSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVHQHTLTVMVRDQDVPVKRNFARIVVNVSDTNDHAPWFTASSYKGRVYESAAVGSVVLQVTALDKDKGKNAEVLYSIESGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGNPINEPLFYHILNPDRRFKISRTSGVLSTTGTPFDREQQEAFDVVVEVTEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNDNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKVLASDADEGSNADITYAIEADSESVKENLEINKLSGVITTKESLIGLENEFFTFFVRAVDNGSPSKESVVLVYVKILPPEMQLPKFSEPFYTFTVSEDVPIGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVLTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSKPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNESLAAPDLSKPRGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV